(19) United States
(12) Patent Application Publication
(10) Pub. No.: US 2013/0190644 A1
(43) Pub. Date: Jul. 25, 2013

(54) ACUPRESSURE GLOVE

(71) Applicant: Donald SPECTOR, New York, NY (US)

(72) Inventor: Donald SPECTOR, New York, NY (US)

(21) Appl. No.: 13/465,425

(22) Filed: May 7, 2012

Related U.S. Application Data
(60) Provisional application No. 61/589,959, filed on Jan. 24, 2012.

(51) Int. Cl. A61B 5/04; A61H 7/00

(52) U.S. Cl. 600/546; 601/135

(57) ABSTRACT

A glove for administering acupressure therapy has a front side having five finger extensions and a pocket at a tip of each of the finger extensions, and a rear side connected to the front side to form a glove with an opening at a bottom edge thereof. There are a plurality of projections that are adapted to be removably placed in each one of the pockets. The projections are of various sizes, so that the acupressure therapist can formulate a glove that is customized to the therapy to be administered.

### CROSS-REFERENCE TO RELATED APPLICATIONS

This application claims priority under 35 U.S.C. 119(e) of U.S. Provisional Application Ser. No. 61/589,959, filed on Jan. 24, 2012.

### BACKGROUND OF THE INVENTION

1. Field of the Invention

The present invention relates to a glove that assists an acupressure therapist in administering acupressure therapy to a patient. In particular, the invention relates to a glove that has interchangeable beads inserted into pockets in the fingertips of the glove, to aid in applying pressure to the patient's acupressure points.

2. The Prior Art

There are several gloves known in the art that assist a therapist in administering massage therapy or acupressure treatment. One such glove is described in U.S. Pat. No. 6,669,657 to Ongwela. This patent describes a massage glove that has hemispherical projections mounted on each of the fingertips to allow increased pressure by the therapist. Other gloves use projections in addition to a motorized device to impart additional massaging features. Such gloves are described in U.S. Pat. No. 6,748,604 to Duboff et al. and in U.S. Patent Application Publication Nos. 2008/0216207 to Tsai and 2005/0143679 to Gelber et al. However, none of these gloves are suitable for precise acupressure treatment, where it is necessary to find the exact acupressure point to be treated, and to apply a very precise amount of pressure.

### SUMMARY OF THE INVENTION

It is therefore an object of the invention to provide a glove that assists a therapist in administering acupressure therapy by locating acupressure points, and by providing an interchangeable set of projections for the fingertips, so that the therapist can set the size of the projection based on the type and location of the acupressure therapy.

The invention comprises a glove for administering acupressure therapy has a front side having five finger extensions and a pocket at a tip of each of the finger extensions, and a rear side connected to the front side to form a glove with an opening at a bottom edge thereof. There are a plurality of projections that are adapted to be removably placed in each one of the pockets. The projections are of various sizes, so that the acupressure therapist can formulate a glove that is customized to the therapy to be administered.

In one embodiment, the projections are spheres. However, other shapes could also be used. The projections preferably have a diameter of 1 mm to 20 mm. The projections can also be magnetic or electromagnetic. The pockets are sized to accommodate all of the different sizes of projections. The pockets can have a fastener as well, to keep the projections inside the pockets. The fastener can be a hook-and-loop-type fastener, i.e, VELCRO®, or another type of fastener, such as a snap or hook.

The glove is preferably made of a breathable, woven fabric. Any suitable material can be used. The projections are preferably made of a hard plastic, but stone, metal or glass could also be used.

In another embodiment, there is an acupressure point detector connected to the glove. The acupressure point detector has sensors connected to each of the tips of the fingers of the front side, so that when a tip passes over an acupressure point on a patient, the detector issues an indicator signal. Acupressure point detectors work by sensing electrical signals in the skin, which indicates an acupressure point. Such detectors are known, but usually consist of a single probe. The present invention incorporates several sensors, by placing one on each fingertip of the glove. This way, when the sensor senses an acupressure point, the detector indicates this to the therapist, either with a visual or audible signal, and the therapist can immediately apply acupressure therapy to the site, using the finger that sensed the acupressure point.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a front view of the glove according to one embodiment of the invention;

FIG. 2 shows a side cross-sectional view of one finger of the glove; and

FIG. 3 shows the glove according to the invention having an acupressure detector mounted therein.

### DETAILED DESCRIPTION OF THE PREFERRED EMBODIMENT

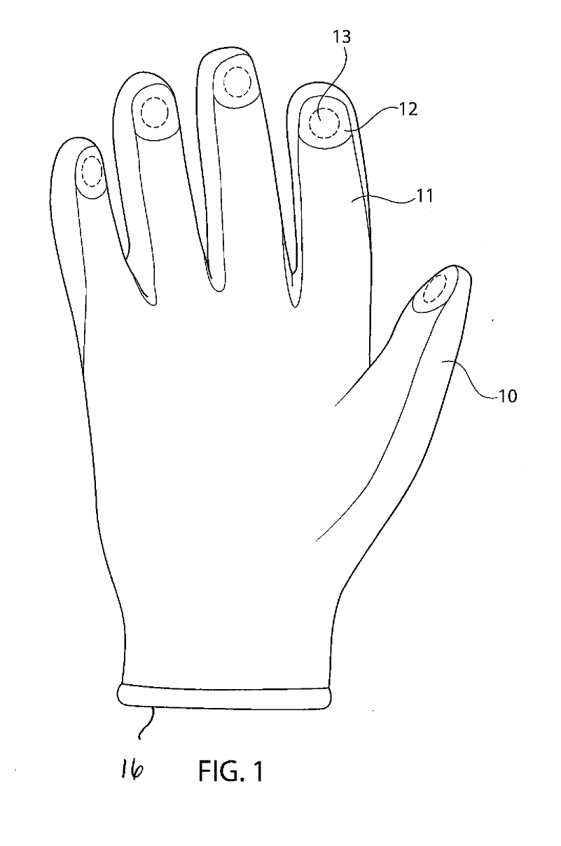

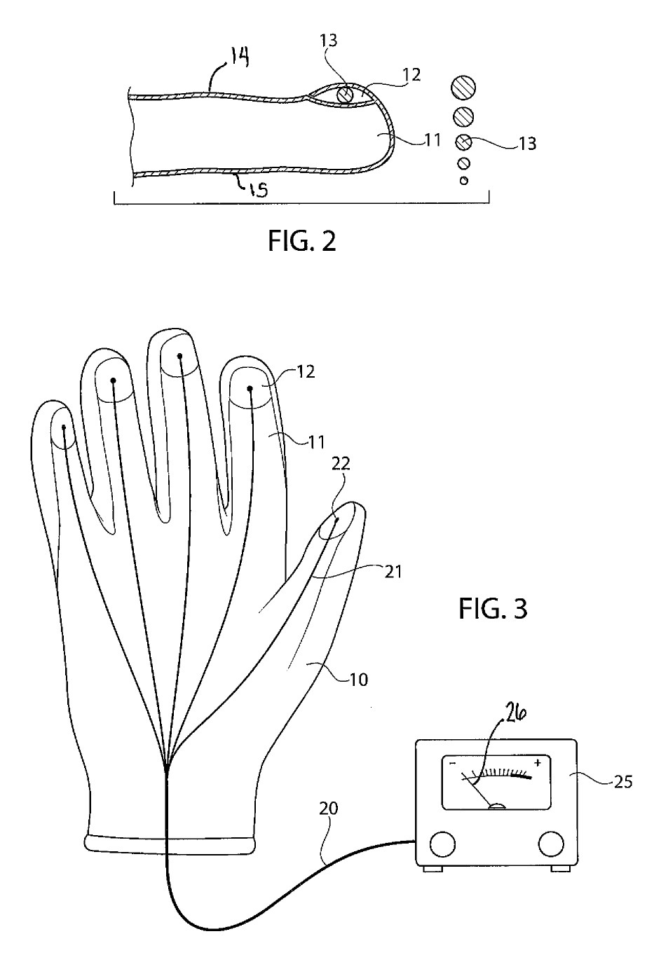

Referring now in detail to the drawings, FIGS. 1 and 2 show a glove 10 for administering acupressure therapy has a front side 14 having five finger extensions 11 and a pocket 12 at a tip of each of the finger extensions, and a rear side 15 connected to the front side to form a glove with an opening at a bottom edge 16. There are a plurality of projections 13 that are adapted to be removably placed in each one of the pockets 12. The projections are of various sizes, so that the acupressure therapist can formulate a glove that is customized to the therapy to be administered.

In this embodiment, projections 13 are spheres. However, other shapes could also be used. The projections preferably have a diameter of 1 mm to 20 mm. The projections can also be magnetic or electromagnetic. The pockets 12 are sized to accommodate all of the different sizes of projections. As shown in FIG. 2, projections 13 are inserted into the pockets 12 to create a raised portion of the glove on the fingertip. This allows the therapist to provide extra pressure when administering acupressure therapy.

In the embodiment shown in FIG. 3, an acupressure point detector 25 connected to the glove 10 via main wire 20 and extension wires 22, which are both attached to glove 10. There are sensors 22 connected to each of the wires 21 at the tips of the fingers of the front side 14, so that when a tip passes over an acupressure point on a patient, the detector 25 issues an indicator signal, in this case, the indicator needle 26 moves from left to right. This way, when the sensor senses an acupressure point, the detector indicates this to the therapist, and the therapist can immediately apply acupressure therapy to the site, using the finger that sensed the acupressure point. The wires 20, 21 and sensors 22 could be removable so that the glove can be used without the detector, and for cleaning purposes. In the event that electromagnetic projections are used in this case, the projections can become magnetized when placed over an acupressure point, due to the electrical activity present there.

Accordingly, while only a few embodiments of the present invention have been shown and described, it is obvious that many changes and modifications may be made thereunto without departing from the spirit and scope of the invention.

What is claimed is:

1. A glove for administering acupressure therapy, comprising:
a front side having five finger extensions and a pocket at a tip of each of the finger extensions;
a rear side with five finger extensions, the rear side being connected to the front side to form a glove with an opening at a bottom edge thereof; and
a plurality of projections for each finger extension, wherein one projection is adapted to be removably placed in each one of the pockets, and wherein the projections are of several different sizes.

2. The glove according to claim 1, wherein the projections are spheres.

3. The glove according to claim 1, wherein the projections have a diameter of 1 mm to 20 mm.

4. The glove according to claim 1, further comprising an acupressure point detector connected to the glove, said acupressure point detector having sensors connected to each of the tips of the fingers of the front side, such that when a tip passes over an acupressure point on a patient, the detector issues an indicator signal.

5. The glove according to claim 1, wherein the projections are magnetic or electromagnetic.
